SEQUENCE LISTING (26 sequences)

SEQ ID NO: 1 - protein, 376 aa, Epstein-Barr virus
MDLDLERGPPGPRRPPRGPPLSSYIALALLLLLLALLFWLYIIMSNWTGGALLVLYAFALMLVIIILIIFIFRRDLLCPLGALCLLLLMITLLLIALWNLHGQALYLGIVLFIFGCLLVLGIWVYFLEILWRLGATIWQLLAFFLAFFLDILLLIIALYLQQNWWTLLVDLLWLLLFLAILIWMYYHGQRHSDEHHHDDSLPHPQQATDDSSNHSDSNSNEGRHHLLVSGAGDAPPLCSQNLGAPGGGPDNGPQDPDNTDDNGPQDPDNTDDNGPHDPLPQDPDNTDDNGPQDPDNTDDNGPHDPLPHNPSDSAGNDGGPPNLTEEVENKGGDRGPPSMTDGGGGDPHLPTLLLGTSGSGGDDDDPHGPVQLSYYD

SEQ ID NO: 2 - protein, 497 aa, Epstein-Barr virus
MGSLEMVPMGAGPPSPGGDPDGDDGGNNSQYPSASGSDGNTPTPPNDEERESNEEPPPPYEDLDWGNGDRHSDYQPLGNQDPSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLTKILLARLFLYALALLLLASALIAGGSILQTNFKSLSSTEFIPNLFCMLLLIVAGILFILAILTEWGSGNRTYGPVFMCLGGLLTMVAGAVWLTVMTNTLLSAWILTAGFLIFLIGFALFGVIRCCRYCCYYCLTLESEERPPTPYRNTV

SEQ ID NO: 3 - protein, 238 aa, Epstein-Barr virus
PFFHPVGEADYFEYLQEGGPDGEPDVPPGAIEQGPADDPGEGPSTGPRGQGDGGRRKKGGWFGKHRGQGGSNPKFENIAEGLRVLLARSHVERTTEEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIKVTVCSFDDGVDLPPWFPPMVEGAAAEGDDGDDGDEGGDGDEGEEGQE

SEQ ID NO: 4 - protein, 9 aa, Epstein-Barr virus
CLGGLLTMV

SEQ ID NO: 5 - protein, 9 aa, Epstein-Barr virus
FLYALALLL

SEQ ID NO: 6 - protein, 9 aa, Epstein-Barr virus
YLQQNWWTL

SEQ ID NO: 7 - protein, 9 aa, Epstein-Barr virus
YLLEMLWRL

SEQ ID NO: 8 - protein, 9 aa, Epstein-Barr virus
ALLVLYSFA

SEQ ID NO: 9 - protein, 9 aa, Epstein-Barr virus
LLSAWILTA

SEQ ID NO: 10 - protein, 9 aa, Epstein-Barr virus
LTAGFLIFL

SEQ ID NO: 11 - protein, 11 aa, Epstein-Barr virus
SSCSSCPLSKI

SEQ ID NO: 12 - protein, 8 aa, Epstein-Barr virus
PYLFWLAA

SEQ ID NO: 13 - protein, 9 aa, Epstein-Barr virus
TYGPVFMCL

SEQ ID NO: 14 - protein, 10 aa, Epstein-Barr virus
VMSNTLLSAW

SEQ ID NO: 15 - protein, 8 aa, Epstein-Barr virus
CPLSKILL

SEQ ID NO: 16 - protein, 9 aa, Epstein-Barr virus
RRRWRRLTV

SEQ ID NO: 17 - protein, 9 aa, Epstein-Barr virus
IEDPPFNSL

SEQ ID NO: 18 - protein, 9 aa, Epstein-Barr virus
IALYLQQNW

SEQ ID NO: 19 - protein, 9 aa, Epstein-Barr virus
MSNTLLSAW

SEQ ID NO: 20 - protein, 9 aa, Epstein-Barr virus
VLKDAIKDL

SEQ ID NO: 21 - protein, 9 aa, Epstein-Barr virus
RPQKRPSCI

SEQ ID NO: 22 - protein, 9 aa, Epstein-Barr virus
IPQCRLTPL

SEQ ID NO: 23 - protein, 9 aa, Epstein-Barr virus
YNLRRGTAL

SEQ ID NO: 24 - protein, 11 aa, Epstein-Barr virus
HPVGEADYFEY

SEQ ID NO: 25 - protein, 9 aa, Epstein-Barr virus
LSRLPFGMA

SEQ ID NO: 26 - protein, 10 aa, Epstein-Barr virus
FVYGGSKTSL

(19) United States
(12) Patent Application Publication
(10) Pub. No.: US 2022/0409662 A1
(43) Pub. Date: Dec. 29, 2022

(54) METHODS OF TREATING AUTOIMMUNE DISEASE USING ALLOGENEIC T CELLS

(71) Applicant: The Council of the Queensland Institute of Medical Research, Herston (AU)

(72) Inventor: Rajiv Khanna, Herston (AU)

(21) Appl. No.: 17/727,107

(22) Filed: Apr. 22, 2022

Related U.S. Application Data
(63) Continuation of application No. 16/303,680, filed on Nov. 21, 2018.
(60) Provisional application No. 62/487,814, filed on Apr. 20, 2017.
(60) Provisional application No. 62/359,326, filed on Jul. 7, 2016.
(60) Provisional application No. 62/341,360, filed on May 25, 2016.

(51) Int. Cl. A61K 35/17; A61P 37/00; C07K 14/74

(52) U.S. Cl. CPC A61K 35/17; A61P 37/00; C07K 14/70539

(57) ABSTRACT

Provided herein are compositions and methods related to the treatment of an autoimmune disease in a subject.

### RELATED APPLICATIONS

This application is a continuation of U.S. patent application Ser. No. 16/303,680, filed on Nov. 21, 2018, which is a § 371 national-stage application based on PCT/IB2017/000805, filed May 25, 2017 which claims the benefit of priority to U.S. Provisional Patent Application Ser. No. 62/341,360 filed May 25, 2016, U.S. Provisional Patent Application Ser. No. 62/359,326, filed on Jul. 7, 2016, and U.S. Provisional Patent Application Ser. No. 62/487,814, filed on Apr. 20, 2017, each of which is incorporated by reference in its entirety.

### SEQUENCE LISTING

The instant application contains a Sequence Listing which has been filed electronically in ASCII format and is hereby incorporated by reference in its entirety. Said ASCII copy, created on Aug. 23, 2022, is named QAH-00902_SL.txt and is 13,522 bytes in size.

### BACKGROUND

Autoimmune diseases, such as multiple sclerosis (MS) and systemic autoimmune disease (SAD), and inflammatory bowel disease (IBD) are pathologies arising from abnormal immune response against the body's own tissue. MS is characterized by the degradation of the myelin, a protective lipid shell surrounding nerve fibers, by the body's own immune cells. SADs are a group of connective tissue diseases with diverse symptoms that include rheumatoid arthritis (RA), systemic lupus erythematosus (SLE) and Sjögren's syndrome (SS). IBDs are a group of inflammatory conditions of the colon and small intestine that include Crohn's disease, celiac disease, and ulcerative colitis.

Epstein Barr Virus (EBV), also known as human herpesvirus 4, is a ubiquitous herpes virus. Recently, it has been shown that exposure to EBV can predispose or otherwise play a role in the pathogenesis of autoimmune diseases, including MS, SAD and IBD. For example, recent studies have shown that individuals diagnosed with MS show higher levels of EBV related proteins in B cells aggregated in nerve tissue than healthy individuals. It is hypothesized that an increase of EBV-infected B cells and/or defective elimination of such cells may predispose individuals to such autoimmune diseases.

### SUMMARY

Provided herein are methods for treating autoimmune diseases (e.g., MS, SAD and/or IBD), comprising administering to a subject allogeneic cytotoxic T cells (CTLs) expressing a T cell receptor that specifically binds to an EBV peptide presented on a class I MHC. In some embodiments, the class I MHC to which the TCR is restricted is encoded by an HLA allele that is present in the subject. In some embodiments, the method comprises selecting the allogeneic CTLs from a cell bank. In some embodiments, the EBV peptide comprises a LMP1 peptide or a fragment thereof, a LMP2A peptide or fragment thereof, and/or an EBNA1 peptide or fragment thereof. In some embodiments, the EBV peptide comprises a sequence listed in Table 1.

In certain aspects, provided herein are methods of treating an autoimmune disease (e.g., MS, SAD and/or IBD), comprising generating allogeneic CTLs that express a T cell receptor that specifically binds to an EBV peptide presented on a class I MHC and then administering the allogeneic CTLs to a subject. In some embodiments, the allogeneic CTLs are stored in a cell bank prior to administration to the subject. In some embodiments, the class I MHC to which the TCR is restricted is encoded by an HLA allele that is present in the subject. In some embodiments, the allogeneic CTLs are generated by incubating a sample comprising allogeneic CTLs (e.g., a PBMC sample) with antigen presenting cells (APCs) presenting an EBV peptide on a class I MHC (e.g., a class I MHC encoded by an HLA allele that is present in the subject), thereby inducing proliferation peptide-specific CTLs in the sample. In some embodiments, the APCs are made to present the EBV peptide by incubating them with a nucleic acid construct (e.g., AdE1-LMPpoly) encoding for the EBV peptide, thereby inducing the APCs to present the EBV peptide. In some embodiments, the APCs may be B cells, antigen-presenting T cells, dendritic cells, or artificial antigen-presenting cells (e.g., a cell line expressing CD80, CD83, 41BB-L and/or CD86, such as aK562 cells). In some embodiments, the EBV peptide comprises a LMP1 peptide or a fragment thereof, a LMP2A peptide or fragment thereof, and/or an EBNA1 peptide or fragment thereof. In some embodiments, the EBV peptide comprises a sequence listed in Table 1.

In some embodiments, CTLs are selected (e.g., selected from a cell bank) for compatibility with the subject prior to administration to the subject. In some embodiments, the CTLs are selected if they are restricted through an HLA allele shared with the subject (i.e., the TCR of the CLTs are restricted to an MHC class I protein encoded by a HLA allele that is present in the subject). In some embodiments, the CTLs are selected if the CTLs and subject share at least 2 (e.g., at least 3, at least 4, at least 5, at least 6) HLA alleles and the CTLs are restricted through a shared HLA allele. In some embodiments, the CTLs administered to the subject are selected from a cell bank (e.g., a CTL bank).

### BRIEF DESCRIPTION OF THE DRAWINGS

FIG. 1 shows improved effector function in CTL product obtained from healthy (NMDP) donors compared to CTL product obtained from MS patients as measured by fraction of viable lymphocytes that are CD8⁺ and IFNg⁺ following stimulation (Mann Whitney p value=0.0002).

### DETAILED DESCRIPTION

### General

Provided herein are methods of treating autoimmune disorders (e.g., MS, SAD and/or IBD) in a subject using allogeneic CTLs that recognize one or more of the EBV epitopes described herein, for example. In some embodiments, the method further comprises selecting the allogeneic CTLs from a cell bank. In some embodiments, the method further comprises making the allogeneic CTLs.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, peptide described herein, an antigen presenting cell provided herein and/or a CTL provided herein.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “binding” or “interacting” refers to an association, which may be a stable association, between two molecules, e.g., between a TCR and a peptide/MHC, due to, for example, electrostatic, hydrophobic, ionic and/or hydrogen-bond interactions under physiological conditions.

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

As used herein, the term “cytokine” refers to any secreted polypeptide that affects the functions of cells and is a molecule which modulates interactions between cells in the immune, inflammatory or hematopoietic response. A cytokine includes, but is not limited to, monokines and lymphokines, regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epidermal keratinocytes and B-lymphocytes. Lymphokines are generally referred to as being produced by lymphocyte cells. Examples of cytokines include, but are not limited to, Interleukin-1 (IL-1), Interleukin-2 (IL-2), Interleukin-6 (IL-6), Interleukin-8 (IL-8), Tumor Necrosis Factor-alpha (TNFα), and Tumor Necrosis Factor beta (TNFβ).

The term “epitope” means a protein determinant capable of specific binding to an antibody or TCR. Epitopes usually consist of chemically active surface groupings of molecules such as amino acids or sugar side chains. Certain epitopes can be defined by a particular sequence of amino acids to which an antibody is capable of binding.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, “specific binding” refers to the ability of a TCR to bind to a peptide presented on an MHC (e.g., class I MHC or class II MHC). Typically, a TCR specifically binds to its peptide/MHC with an affinity of at least a K_{D} of about 10⁻⁴ M or less, and binds to the predetermined antigen/binding partner with an affinity (as expressed by K_{D}) that is at least 10 fold less, at least 100 fold less or at least 1000 fold less than its affinity for binding to a non-specific and unrelated peptide/MHC complex (e.g., one comprising a BSA peptide or a casein peptide).

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

As used herein, the term “treating” a disease in a subject or “treating” a subject having or suspected of having a disease refers to subjecting the subject to a pharmaceutical treatment, e.g., the administration a CTL described herein, such that at least one symptom of the disease is decreased or prevented from worsening.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that may or may not be able to replicate autonomously or integrate into a chromosome of a host cell.

### Peptides

In certain aspects, provided herein are methods of treating autoimmune disorders (e.g., MS, SAD and/or IBD) using allogeneic CTLs expressing TCRs that specifically bind to peptides comprising EBV epitopes presented on class I MHC. In some embodiments, provided herein are methods generating such allogeneic CTLs, for example, by incubating a sample comprising CTLs (i.e., a PBMC sample) with antigen-presenting cells (APCs) that present one or more of the EBV epitopes described herein (e.g., APCs that present a peptide described herein comprising a EBV epitope on a class I MHC complex).

In some embodiments, the peptides provided herein comprise a sequence of any EBV viral protein (e.g., a sequence of at least 5, 6, 7, 8, 9, 10, 11, 12, 13, 14, 15, 16, 17, 18, 19 or 20 contiguous amino acids of any EBV protein). In some embodiments, the peptides provided herein comprise no more than 25, 20, 19, 18, 17, 16, 15, 14, 13, 12, 11 or 10 contiguous amino acids of the EBV viral protein.

In some embodiments, the peptides provided herein comprise a sequence of LMP1 (e.g., a sequence of at least 5, 6, 7, 8, 9, 10, 11, 12, 13, 14, 15, 16, 17, 18, 19 or 20 contiguous amino acids of LMP1). In some embodiments, the peptides provided herein comprise no more than 25, 20, 19, 18, 17, 16, 15, 14, 13, 12, 11 or 10 contiguous amino acids of LMP1. An exemplary LMP1 amino acid sequence is provided below (SEQ ID NO: 1):

| | |
|---|---|
| 1 | mdldlergpp gprrpprgpp Issyialall llllallfwl yiimsnwtgg allvlyafal |
| 61 | mlviiiliif ifrrdllcpl galcllllmi tlllialwnl hgqalylgiv ififgcllvl |
| 121 | giwvyfleil wrlgatiwql lafflaffld illliialyl qqnwwtllvd llwlllflai |
| 181 | liwmyyhgqr hsdehhhdds lphpqqatdd ssnhsdsnsn egrhhllvsg agdapplcsq |
| 241 | nlgapgggpd ngpqdpdntd dngpqdpdnt ddngphdplp qdpdntddng pqdpdntddn |
| 301 | gphdplphnp sdsagndggp pnlteevenk ggdrgppsmt dggggdphlp tlllgtsgsg |
| 361 | gddddphgpv qlsyyd |

In some embodiments, the peptides provided herein comprise a sequence of LMP2A (e.g., a sequence of at least 5, 6, 7, 8, 9, 10, 11, 12, 13, 14, 15, 16, 17, 18, 19 or 20 contiguous amino acids of LMP2A). In some embodiments, the peptides provided herein comprise no more than 25, 20, 19, 18, 17, 16, 15, 14, 13, 12, 11 or 10 contiguous amino acids of LMP2A. An exemplary LMP2A amino acid sequence is provided below (SEQ ID NO: 2):

| | |
|---|---|
| 1 | mgslemvpmg agppspggdp dgddggnnsq ypsasgsdgn tptppndeer esneeppppy |
| 61 | edldwgngdr hsdyqplgnq dpslylglqh dgndglpppp ysprddssqh iyeeagrgsm |
| 121 | npvclpviva pylfwlaaia ascftasvst vvtatglals llllaavass yaaaqrkllt |
| 181 | pvtvitavvt ffaicltwri edppfnsllf allaaagglq giyvlvmlvl lilayrrrwr |
| 241 | rltvcggimf lacvivlivd avlqlspllg avtvvsmtll llafvlwlss pgglgtlgaa |
| 301 | lltlaaalal laslilgtin ittmfllmll wtlvvllics scsscpltki llarlflyal |
| 361 | allllasali aggsilqtnf kslsstefip nlfcmllliv agilfilail tewgsgnrty |
| 421 | gpvfmclggl ltmvagavwl tvmtntllsa wiltagflif ligfalfgvi rccryccyyc |
| 481 | itleseerpp tpyrntv |

In some embodiments, the peptides provided herein comprise a sequence of EBNA1 (e.g., a sequence of at least 5, 6, 7, 8, 9, 10, 11, 12, 13, 14, 15, 16, 17, 18, 19 or 20 contiguous amino acids of EBNA1). In some embodiments, the peptides provided herein comprise no more than 25, 20, 19, 18, 17, 16, 15, 14, 13, 12, 11 or 10 contiguous amino acids of EBNA1. An exemplary EBNA1 amino acid sequence is provided below (SEQ ID NO: 3):

```
    1 pffhpvgead yfeylqeggp dgepdvppga ieqgpaddpg egpstgprgq gdggrrkkgg
  61 wfgkhrgqgg snpkfeniae glrvllarsh vertteegtw vagvfvyggs ktslynlrrg
121 talaipqcrl tplsrlpfgm apgpgpqpgp iresivcyfm vfiqthifae vlkdaikdlv
181 mtkpaptcni kvtvcsfddg vdlppwfppm vegaaaegdd gddgdeggdg degeegqe
```

In some embodiments, the peptide comprises the sequence of an epitope listed in Table 1.

TABLE 1
Exemplary EBV viral protein epitopes

| Epitope Sequence | HLA Restriction | SEQ ID NO. |
|---|---|---|
| CLGGLLTMV | A*02 | 4 |
| FLYALALLL | A*02 | 5 |
| YLQQNWWTL | A*02, A*68, A*69 | 6 |
| YLLEMLWRL | A*02 | 7 |
| ALLVLYSFA | A*02 | 8 |
| LLSAWILTA | A*0203 | 9 |
| LTAGFLIFL | A*0206 | 10 |
| SSCSSCPLSKI | A*11 | 11 |
| PYLFWLAA | A*23, A*24, A*30 | 12 |
| TYGPVFMCL | A*24 | 13 |
| VMSNTLLSAW | A*25 | 14 |
| CPLSKILL | B*08 | 15 |
| RRRWRRLTV | B*27 | 16 |
| IEDPPFNSL | B*40 | 17 |
| IALYLQQNW | B*57, B*58 | 18 |
| MSNTLLSAW | B*58 | 19 |
| VLKDAIKDL | A*0203 | 20 |
| RPQKRPSCI | B*07 | 21 |
| IPQCRLTPL | B*07 | 22 |
| YNLRRGTAL | B*08 | 23 |
| HPVGEADYFEY | B*35 | 24 |
| LSRLPFGMA | B*57 | 25 |
| FVYGGSKTSL | Cw*03 | 26 |

In some embodiments, the peptides provided herein comprise two or more of the EBV epitopes. In some embodiments, the peptides provided herein comprise at least 2, 3, 4, 5, 6, 7, 8, 9, 10, 11, 12, 13, 14, 15, 16, 17, 18, 19 or 20 EBV epitopes. For example, in some embodiments, the peptide provided herein comprises two or more of the EBV epitopes connected by linkers (e.g., polypeptide linkers).

In some embodiments, the sequence of the peptides comprises an EBV viral protein sequence except for 1 or more (e.g., 1, 2, 3, 4, 5, 6, 7, 8, 9, 10 or more) conservative sequence modifications. As used herein, the term “conservative sequence modifications” is intended to refer to amino acid modifications that do not significantly affect or alter the interaction between a TCR and a peptide containing the amino acid sequence presented on an MHC. Such conservative modifications include amino acid substitutions, additions (e.g., additions of amino acids to the N or C terminus of the peptide) and deletions (e.g., deletions of amino acids from the N or C terminus of the peptide). Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues of the peptides described herein can be replaced with other amino acid residues from the same side chain family and the altered peptide can be tested for retention of TCR binding using methods known in the art. Modifications can be introduced into an antibody by standard techniques known in the art, such as site-directed mutagenesis and PCR-mediated mutagenesis.

In some embodiments, the peptides provided herein comprise a sequence that is at least 80%, 85%, 90%, 95% or 100% identical to an EBV viral protein sequence (e.g., the sequence of a fragment of an EBV viral protein). To determine the percent identity of two amino acid sequences, the sequences are aligned for optimal comparison purposes (e.g., gaps can be introduced in one or both of a first and a second amino acid sequence for optimal alignment and non-identical sequences can be disregarded for comparison purposes). The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

In some embodiments, the peptide is chimeric or fusion peptide. As used herein, a “chimeric peptide” or “fusion peptide” comprises a peptide having a sequence provided herein linked to a distinct peptide having sequence to which it is not linked in nature. For example, the distinct peptide can be fused to the N-terminus or C-terminus of the peptide provided herein either directly, through a peptide bond, or indirectly through a chemical linker. In some embodiments, the peptide of the provided herein is linked to another peptide comprising a distinct EBV epitopes. In some embodiments, the peptide provided herein is linked to peptides comprising epitopes from other viral and/or infectious diseases.

A chimeric or fusion peptide provided herein can be produced by standard recombinant DNA techniques. For example, DNA fragments coding for the different peptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, for example, Current Protocols in Molecular Biology, Ausubel et al., eds., John Wiley & Sons: 1992). Moreover, many expression vectors are commercially available that already encode a fusion moiety.

The peptides provided herein can be isolated from cells or tissue sources by an appropriate purification scheme using standard protein purification techniques, and can be produced by recombinant DNA techniques, and/or can be chemically synthesized using standard peptide synthesis techniques. The peptides described herein can be produced in prokaryotic or eukaryotic host cells by expression of nucleotides encoding a peptide(s) of the present invention. Alternatively, such peptides can be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N. Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

In certain aspects, provided herein are nucleic acid molecules encoding the peptides described herein. In some embodiments, the nucleic acid molecule is a vector. In some embodiments, the nucleic acid molecule is a viral vector, such as an adenovirus based expression vector, that comprises the nucleic acid molecules described herein. In some embodiments, the vector provided herein encodes a plurality of epitopes provided herein (e.g., as a polyepitope). In some embodiments, the vector provided herein encodes at least 2, 3, 4, 5, 6, 7, 8, 9, 10, 11, 12, 13, 14, 15, 16, 17, 18, 19 or 20 epitopes provided herein (e.g., epitopes provided in Table 1).

In some embodiments, the vector is AdE1-LMPpoly. The AdE1-LMPpoly vector encodes a polyepitope of defined CTL epitopes from LMP1 and LMP2 fused to a Gly-Ala repeat-depleted EBNA1 sequence. The AdE1-LMPpoly vector is described, for example, in Smith et al., Cancer Research 72:1116 (2012); Duraiswamy et al., Cancer Research 64:1483-9 (2004); and Smith et al., J. Immunol 117:4897-906, each of which is hereby incorporated by reference.

As used herein, the term “vector,” refers to a nucleic acid molecule capable of transporting another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication, episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”). In some embodiments, provided herein are nucleic acids operably linked to one or more regulatory sequences (e.g., a promotor) in an expression vector. In some embodiments, the cell transcribes the nucleic acid provided herein and thereby expresses a peptide described herein. The nucleic acid molecule can be integrated into the genome of the cell or it can be extrachromasomal.

In some embodiments, provided herein are cells that contain a nucleic acid described herein (e.g., a nucleic acid encoding a peptide described herein). The cell can be, for example, prokaryotic, eukaryotic, mammalian, avian, murine and/or human. In some embodiments, the cell is a mammalian cell. In some embodiments the cell is an APC (e.g. an antigen-presenting T cell, a dendritic cell, a B cell, or an aK562 cell). In the present methods, a nucleic acid described herein can be administered to the cell, for example, as nucleic acid without delivery vehicle, in combination with a delivery reagent. In some embodiments, any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. In some embodiments of the methods described herein, liposomes are used to deliver a nucleic acid to a cell or subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

### Allogeneic CTLs

Provided herein are methods of treating autoimmune diseases (e.g., MS, SAD, IBD) by administering to the subject allogeneic CTLs expressing a T cell receptor that specifically binds to an EBV peptide presented on a class I MHC. In some embodiments, the CTLs are from a cell bank. In some embodiments, the MHC is a class I MHC. In some embodiment, the class II MHC has an α chain polypeptide that is HLA-DMA, HLA-DOA, HLA-DPA, HLA-DQA or HLA-DRA. In some embodiments, the class II MHC has a (3 chain polypeptide that is HLA-DMB, HLA-DOB, HLA-DPB, HLA-DQB or HLA-DRB. In some embodiments, the CTLs are stored in a cell library or bank before they are administered to the subject.

In some embodiments, provided herein are APCs that present a peptide described herein (e.g., a peptide comprising a LMP1, LMP2A, or EBNA1 epitope sequence). In some embodiments the APCs are B cells, antigen presenting T-cells, dendritic cells, or artificial antigen-presenting cells (e.g., aK562 cells).

Dendritic cells for use in the process may be prepared by taking PBMCs from a patient sample and adhering them to plastic. Generally, the monocyte population sticks and all other cells can be washed off. The adherent population is then differentiated with IL-4 and GM-CSF to produce monocyte derived dendritic cells. These cells may be matured by the addition of IL-1β, IL-6, PGE-1 and TNF-α (which upregulates the important co-stimulatory molecules on the surface of the dendritic cell) and are then transduced with one or more of the peptides provided herein.

In some embodiments, the APC is an artificial antigen-presenting cell, such as an aK562 cell. In some embodiments, the artificial antigen-presenting cells are engineered to express CD80, CD83, 41BB-L, and/or CD86. Exemplary artificial antigen-presenting cells, including aK562 cells, are described U.S. Pat. Pub. No. 2003/0147869, which is hereby incorporated by reference.

In certain aspects, provided herein are methods of generating APCs that present the one or more of the EBV epitopes described herein comprising contacting an APC with a peptide comprising a EBV epitope and/or with a nucleic acid encoding a EBV epitope. In some embodiments, the APCs are irradiated. In some embodiments, the APCs that present a peptide described herein (e.g., a peptide comprising a LMP1, LMP2A, or EBNA1 epitope sequence). A cell presenting a peptide described herein can be produced by standard techniques known in the art. For example, a cell may be pulsed to encourage peptide uptake. In some embodiments, the cells are transfected with a nucleic acid encoding a peptide provided herein. Provided herein are methods of producing antigen-presenting cells (APCs), comprising pulsing a cell with the peptides described herein. Exemplary examples of producing antigen presenting cells can be found in WO2013088114, hereby incorporated in its entirety.

In some embodiments, provided herein are T cells (e.g., CD4 T cells and/or CD8 T cells) that express a TCR (e.g., an αβ TCR or a γδ TCR) that recognizes a peptide described herein presented on a MHC. In some embodiments, the T cell is a CD8 T cell (a CTL) that expresses a TCR that recognizes a peptide described herein presented on a class I MHC. In some embodiments, the T cell is a CD4 T cell (a helper T cell) that recognizes a peptide described herein presented on a class II MHC.

In some embodiments, provided herein are methods of generating, activating and/or inducing proliferation of T cells (e.g., CTLs) that recognize one or more of the EBV epitopes described herein. In some embodiments, a sample comprising CTLs (i.e., a PBMC sample) is incubated in culture with an APC provided herein (e.g., an APC that presents a peptide comprising a EBV epitope on a class I MHC complex). In some embodiments, the APCs are autologous to the subject from whom the T cells were obtained. In some embodiments, the APCs are not autologous to the subject from whom the T cells were obtained. In some embodiments, the sample containing T cells are incubated 2 or more times with APCs provided herein. In some embodiments, the T cells are incubated with the APCs in the presence of at least one cytokine. In some embodiments, the cytokine is IL-4, IL-7 and/or IL-15. Exemplary methods for inducing proliferation of T cells using APCs are provided, for example, in U.S. Pat. Pub. No. 2015/0017723, which is hereby incorporated by reference.

In some embodiments, provided herein are compositions (e.g., therapeutic compositions) comprising T cells and/or APCs provided herein used to treat and/or prevent an autoimmune disease in a subject by administering to the subject an effective amount of the composition. In some aspects, provided herein are methods of treating autoimmune disorders using a composition (e.g., a pharmaceutical composition, such compositions comprising allogeneic CTLs). In some embodiments, the composition includes a combination of multiple (e.g., two or more) CTLs provided herein.

### Therapeutic Methods

In some embodiments, the provided herein are methods of treating an autoimmune disorder in a subject by administering to the subject allogeneic CTLs provided herein. In some embodiments, the allogenic CTLs are selected from a cell bank (e.g., a pre-generated third party donor derived bank of epitope-specific CTLs).

In some embodiments, the methods provided herein can be used to treat any autoimmune disease. Examples of autoimmune diseases include, for example, glomerular nephritis, arthritis, dilated cardiomyopathy-like disease, ulceous colitis, Sjogren syndrome, Crohn disease, systemic erythematodes, chronic rheumatoid arthritis, juvenile rheumatoid arthritis, Still's disease, multiple sclerosis, psoriasis, allergic contact dermatitis, polymyositis, pachyderma, periarteritis nodosa, rheumatic fever, vitiligo vulgaris, Behcet disease, Hashimoto disease, Addison disease, dermatomyositis, myasthenia gravis, Reiter syndrome, Graves' disease, anaemia perniciosa, sterility disease, pemphigus, autoimmune thrombopenic purpura, autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus erythematosus, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, lymphopenia, mixed connective tissue disease, pemphigoid, pemphigus vulgaris, pernicious anemia, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, type I diabetes, ulcerative colitis and Wegener's granulomatosis.

In some embodiments, the methods provided herein are used to treat MS. In some embodiments, the MS is relapsing-remitting MS, secondary progressive MS, primary progressive MS or progressively relapsing MS.

In some embodiments, the methods provided herein are used to treat a SAD. For example, in certain embodiments, the methods provided herein are used to treat rheumatoid arthritis, systemic lupus erythematosus and/or Sjögren's syndrome.

In some embodiments, the methods provided herein are used to treat IBD. For example, in certain embodiments the methods provided herein are used to treat Crohn's disease (regional bowel disease, e.g., inactive and active forms), celiac disease (e.g., inactive or active forms) and/or ulcerative colitis (e.g., inactive and active forms). In some embodiments, the methods provided herein are used to treat irritable bowel syndrome, microscopic colitis, lymphocytic-plasmocytic enteritis, coeliac disease, collagenous colitis, lymphocytic colitis, eosinophilic enterocolitis, indeterminate colitis, infectious colitis (viral, bacterial or protozoan, e.g. amoebic colitis) (e.g., Clostridium dificile colitis), pseudomembranous colitis (necrotizing colitis), ischemic inflammatory bowel disease, Behcet's disease, sarcoidosis, scleroderma, IBD-associated dysplasia, dysplasia associated masses or lesions, and/or primary sclerosing cholangitis.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

In some embodiments, the method includes selecting allogeneic CTLs from a cell bank (e.g., a pre-generated third party donor derived bank of epitope specific CTLs). In some embodiments, the CTLs are selected because they express a TCR restricted to a class I MHC that is encoded by an HLA allele that is present in the subject. In some embodiments, the CTLs are selected if the CTLs and subject share at least 2 (e.g., at least 3, at least 4, at least 5, at least 6) HLA alleles and the CTLs are restricted through a shared HLA allele. In some embodiments, the method comprises testing the TCR repertoire of the pre-generated third-party-donor-derived epitope-specific T cells (i.e., allogeneic T cells) with flow cytometry. In some embodiments epitope-specific T cells are detected using a tetramer assay, an ELISA assay, a western blot assay, a fluorescent microscopy assay, an Edman degradation assay and/or a mass spectrometry assay (e.g., protein sequencing). In some embodiments, the TCR repertoire is analyzed using a nucleic acid probe, a nucleic acid amplification assay and/or a sequencing assay.

### EXAMPLES

### Example 1: Generating a Third Party Donor Derived Bank of Epitope Specific CTLs

A third party donor derived bank of epitope specific CTLs is generated through the targeted identification of donor lymphocyte material in order to generate CTL populations with sufficient scale, breadth of patient HLA matching capability, and target-restricted activity. Identification of donor material is facilitated by any combination of donor/material genetic annotation or resultant product quality characteristics yielded from the following materials:
(a) Donor HLA alleles—specific HLA alleles may be prioritized and specifically gathered as input material for CTL generation based on ability to cover most broadly the targeted patient population and/or the cognate epitopes contained in the stimulating peptide sequences.
(b) Capacity for a test aliquot of donor material to expand and/or produce effective cytotoxic capacity following the CTL stimulation protocol.
(c) Epitope/HLA restriction of stimulated donor material as indicated by cytotoxicity studies, or through functional characterization of response as indicated by degranulation, cytokine release, signaling assays, or other markers of apoptosis in target cells and/or epitope-specific stimulation of the CTL compartment.
(d) Resultant phenotypic profile of CTL product in a test aliquot as related to expression of co-stimulatory molecules, exhaustion markers, differentiation markers, and/or other resultant product characteristics.

Following the parallel or sequential stimulation and generation of CTL comprising products, each CTL batch or lot is characterized and annotated for HLA restriction specificity and potency. Characterized lots are then viably cryo-preserved to allow for reanimation at a later date. The cumulative cryo-preservation of multiple lots generated from distinct donor material with distinct HLA allele expression results in a breadth of diversity in HLA restricted activity across the cryo-preserved “bank.” The content of the bank is then ready to be selected and matched to patient characteristics at a future date, such that specific lots can be retrieved and reanimated for the purposes of providing a readily-available therapy with characteristics tailored to that of each patient.

### Example 2: Selecting CTLs from a Third Party Donor Cell Line Derived from a Bank of Epitope Specific CTLs

Patient-specific requisitioning of banked products can be accomplished through the ordered and prioritized integration of material characteristics with the patient's genetic or disease background. Such a sequence of hierarchical considerations may be accomplished through use of an algorithm designed to integrate these inputs and output a matching lot. This algorithm can be based on HLA restriction, or, when multiple lots are available, matching by HLA restriction in combination with a series of additional inputs, each appropriately weighted and including additional lot and/or patient-specific characteristics and annotations to select the most effective patient-specific lot, or one that most mitigates potential for adverse events. Below is provided an exemplary format for such a requisitioning algorithm.

Allogeneic third party EBV-CTLs are selected for the subject from the library of available EBV-CTL cell lines. The following steps outline the process to identify the cell line(s) to be used for a subject:

1) In order to match a cell line with a patient, a cell line and patient must share ≥2 HLA loci at high resolution, with at least 1 HLA locus of the subject or preferentially the subject's EBV+B-cell compartment, if known, matched to the given CTL cell line's HLA restriction.

2) In order to ascertain the presence of an adequate cell dose, the presence of sufficient cells from the selected cell line to administer at minimum X cycles at Y CTL/kg actual body weight per dose (n doses per cycle(X), X cycles=nX doses total; therefore, the minimum dose available must be at least nXY×10⁶ CTL/kg actual body weight). The minimum dose may change depending on patient or disease characteristics.

3) If only one cell line is identified according to the previously discussed standards, then that cell line should be used and no further selection criteria are imposed. However, in some cases, there may exist more than one cell line in the CTL library meeting HLA matching (1) and minimum dose requirements (2) for a given subject. Among those CTL cell lines, some may have additional HLA allele characteristics, either restricting or defined in the genotype of the material donor, that may be associated, either clinically or through indirect levels of evidence, with decreased clinical performance, as defined by decreased efficacy or increased association with adverse events. If this is the case, cell lines would be selected for the lack of this additional HLA allele.

Additionally, cell lines that have been previously administered to patients and the resulting responses recorded. These cell line response data is used to select among CTL cell line options meeting the requirements of (1) and (2) as follows:

3a) Among cell lines where the previous response rates have been greater than a specified cut-off among at least 4 patients treated, then the largest existing cell dose available in the library is selected. If the donor starting material was used for a subsequent batch and the same HLA restriction was obtained as for the first batch, then the response rates for the subsequent batches sharing the same HLA restriction can be assumed to be similarly effective.

3b) If there are no cell lines meeting the criteria in (3a), then among the cell lines meeting the requirements of (1) and (2), the line with the highest response rate and at least one previous response is selected.

3c) If there are no cell lines with a previous response rate, select among cell lines whose HLA restriction has been shown previously to elicit responses, prioritizing which cell line by the HLA restriction shared with the subject (or subject's disease) with the highest previous response.

3d) Finally, if previous requirements cannot be met, the line avoiding HLA restrictions with known inadequate response or with increased prevalence or potential association with decreased clinical performance is selected.

Any patient diagnosed with primary progressive MS (PPMS), secondary progressive MS (SPMS), or relapsing remitting MS (RRMS) patients may be treated with EBV-CTLs as long as there is an available cell line with an HLA restriction that matches an HLA allele on the patient.

### Example 3: Treatment of MS Using Third Party Donor Derived CTLs

Patients with relapsing remitting, primary progressive, and secondary progressive MS are treated with third party allogeneic targeted EBV-CTLs that exhibit cytotoxicity against B-cells and plasma cells presenting EBNA1, LMP1, and LMP2 antigens. Patients receive four administrations of targeted EBV-CTLs at a dose of 2×10\hat{ }7 cells/m2, administered intravenously at Q2 week intervals (i.e. on Days, 1, 15, 29, and 43). Patients are assessed for relapse events, serial Gadolinium enhanced brain MRI, and serial lumbar puncture to measure cerebrospinal fluid IgG levels and incidence of oligoclonal bands. The Expanded Disability Status Scale (EDSS) is administered to characterize the progression of disability. Concomitant medications and adverse events are collected to characterize the safety profile of treatment. The following are indications of efficacy of treatment:

1) Significant decreases in new Gadolinium enhancing lesions, as observed on MRI imaging at monthly visits in RRMS patients when compared to historical controls in a similar patient population.

2) Significant decreases in annualized clinical relapse rates at monthly visits when compared to historical controls in a similar patient population.

3) Significant reduction in CSF IgG levels when compared to baseline in primary progressive MS (PPMS), secondary progressive MS (SPMS) and relapsing remitting MS (RRMS) patients.

4) Thirty percent of primary progressive, secondary progressive and relapsing remitting MS patients resolve oligoclonal bands which had been present at baseline.

5) Mild to moderate improvement in EDSS scores at 6 and 12 months in primary progressive MS (PPMS), secondary progressive MS (SPMS) and relapsing remitting MS (RRMS) patients.

6) Significant improvement occurring in motor strength in 50% of RRMS, 30% of PPMS, and 25% of SPMS patients.

7) 80% of RRMS patients showing no evidence of disease activity at 1 year compared to historical controls which showed 65%.

### Example 4: Treatment of MS Using Third Party Donor Derived CTLs (ATA188)

Patients with relapsing remitting, primary progressive, and secondary progressive MS are treated with adoptive transfer of third party donor derived CTLs. Allogeneic latency-2 EBV-targeted cytotoxic T lymphocytes (allogeneic L2 EBV CTLs), or ATA188, are HLA-matched, in vitro-expanded, antigen-specific T cells specific for EBV protein antigens including latent membrane protein 1 (LMP1), LMP2, and EBNA1. ATA188 is produced from the peripheral blood mononuclear cells (PBMCs) of healthy EBV seropositive donors. A portion of these donor cells become the T cells for immunotherapy and a portion are the antigen presenting cells (APC) which are used to stimulate the T cells. The APCs are transduced with a novel, recombinant, replication-deficient adenovirus encoding a transgene that expresses a polypeptide protein and truncated EBNA1 protein (AdE1-LMPpoly). The polyepitope protein includes multiple HLA class I-restricted CD8+ T cell epitopes from LMP1 and LMP2 as a “string of beads”. The truncated EBNA1 protein excludes the glycine-alanine repeat sequence which interferes with translation and endogenous processing of this protein and maintains the CD8+ and CD4+ T cell epitopes. Preclinical and clinical studies have shown that these LMP and EBNA1 expressing APCs are highly effective at inducing the rapid expansion of antigen-specific T cells from human donors in the presence of interleukin-2 (IL-2). The resulting cell product, ATA188, is cryopreserved and verified to be HLA-restricted with cytotoxic potential and to be without adenovirus infectivity.

### Protocol and Dosing

Patients receive 2 cycles of treatment with each cycle consisting of a 15-day treatment period (with 3 infusions, each given approximately 7 days apart, on Days 1, 8 [±2 days], and 15 [±2 days]). After the third infusion of Cycle 1, subjects enter a 20-day observation period with approximately weekly visits, and after the third infusion of Cycle 2, subjects enter a follow-up period with 11 monthly (every 28±5 days) visits. Together, subjects are observed for at least 1 year after the first dose of ATA188.

The first cohort is treated at a dose of 5×10⁶ cells, followed by doses of 1×10⁷, 2.0×10⁷, and 4.0×10⁷, (in Cohorts 2, 3, and 4, respectively). Within Cohorts 1 to 4, treatment is staggered for the subjects, with an 8-day pause between treatment of the first and second subjects and the second and third subjects (e.g., treatment for the second subject may begin the day after the first subject receives their Day 8 infusion, if no dose limiting toxicities are observed. Dose limiting toxicities, or DLTs, is a toxicity considered at least possibly related to the administration of ATA188. Once the third subject is enrolled, the remainder of the cohort is enrolled. Dose escalation from one cohort to the next will occur if no DLTs occur during the first 35 days after the first dose of Cycle 1 Day 1 (i.e., 35-day DLT assessment window) for all 6 subjects in the cohort. If one subject among the six experiences a DLT within the 35-day assessment window, an additional 3 subjects will be enrolled into that dose cohort. If no DLTs are observed (within the 35-day assessment window) among the additional 3 subjects, dose escalation to the next dose cohort will proceed. If 2 or more of the 9 subjects within a cohort experience DLTs within the 35-day assessment window, that dose level will be considered the maximum tolerated dose (MTD). The MTD is highest dose studied at which <1 in 6 subjects have DLT. If all doses have <1 in 6, then the MTD is the highest dose studied. In addition, the previous dose level will be considered the RP2D. RP2D is the ATA188 dose selected for phase 2 based on evaluation of all safety, efficacy, and biomarker data collected during dose escalation (i.e., Cohorts 1-4) with a subject incidence of DLTs of <16.6% during the first 35 days of dosing by the enrolling investigators and sponsor's medical monitor. If 2 or more of the 9 subjects within the lowest dose cohort (Cohort 1) experience DLTs within the 35-day assessment window, a lower dose/schedule may be explored in consultation with the sponsor's medical monitor and the enrolling investigators.

Dose escalation will be based on safety assessments, including treatment-emergent adverse events (TEAEs), clinical laboratory data, physical examination findings, including vital signs, and electrocardiograms (ECGs) after all subjects within a cohort have completed the 35-day DLT assessment window.

The dose expansion (i.e., Cohort 5) will be performed at the RP2D with no staggering/pausing of treatment between subjects.

Patients are assessed for relapse events and change from baseline in the number of gadolinium (Gd)-enhancing and new or enlarging T2 lesions on brain magnetic resonance imaging (MRI) scans. ATA188 is selected for each subject based on matching at least 2 human leukocyte antigen (HLA) alleles with at least 1 HLA-restricting allele shared between ATA188 and the subject. The Expanded Disability Status Scale (EDSS) is administered to characterize the progression of the disease and of disability. Concomitant medications and adverse events are collected to characterize the safety profile of treatment.

### Outcome Measures/Study Assessments:

The following are indications of efficacy of treatment:

1) The change from baseline in the number of Gd-enhancing and new or enlarging T2 lesions on brain MRI scans.

2) Decreases in annualized clinical relapse rates.

3) Mild to moderate improvement in EDSS scores in primary progressive MS (PPMS), secondary progressive MS (SPMS) and relapsing remitting MS (RRMS) patients.

Patients are evaluated for the frequency, persistence, and expansion of circulating EBV-specific T cells, and to correlate cellular kinetics with efficacy and safety endpoints. Additionally, any number of endpoints may be evaluated in the study participants. For example, the change in EBV-deoxyribonucleic acid (DNA), the change in vitamin D3, the change in nuerofilamints, the change in MRI magnetic field transfer ratio (MTR), the change in clinical outcome assessments (e.g., Multiple Sclerosis Impact Scale 29 (MSIS) score, Fatigue Severity Scale (FSS) score, Visual Acuity (VA), and Multiple Sclerosis Functional Composite (MSFC) score)), and the change in immunoglobulin G (IgG) index (including quantification of IgG and oligoclonal band (OCB) analysis in serum and cerebral spinal fluid (CSF)) may be measured by taking a first measurement prior to the administration of T cells and taking additional measurements during or after the study.

### Study Population:

Up to 42 subjects with RRMS and 6 subjects SPMS with recent disease activity will be enrolled at 6 to 10 study sites. If no DLT occurs in the study, a total of 36 subjects will be enrolled (30 with RRMS and 6 with SPMS).

The following are inclusion/exclusion criteria for patients involved in the study. A subject will be considered eligible to participate in this study if all of the following are satisfied:
1. History of MS, meeting one of the following criteria:
   RRMS, as defined by the 2010 Revised McDonald criteria for the diagnosis of MS
OR
   SPMS diagnosed at least 1 year prior to enrollment, with no history of relapses within the year before providing informed consent
2. Positive EBV serology
3. Availability of appropriate partial HLA-matched and restricted ATA188
4. Males and females 18 to 45 years of age
5. EDSS score of 3.0 to 6.5
6. Willing and able to provide written informed consent

A subject will not be eligible to participate in the study if any of the following criteria are met:
1. Concurrent serious uncontrolled or unresolved medical condition, such as infection, limiting protocol compliance or exposing the subject to unacceptable risk
2. Positive serology and/or nucleic acid testing (NAT) for human immunodeficiency virus (HIV)
3. Serology and/or NAT indicating active hepatitis B virus (HBV) infection or carrier status for HBV (Note: A positive serology for HBV indicating a previous but cleared infection with HBV is not an exclusion criterion)
4. Serology and/or NAT indicating active hepatitis C virus (HCV) infection
5. Positive serology for syphilis or human T cell lymphotrophic virus I/II (HTLV)
6. Significant non-malignant disease (eg, severe cardiac or respiratory dysfunction)
7. Uncontrolled psychosis, uncontrolled depression or suicide risk, substance dependence, or any other psychiatric condition that may compromise the ability to participate in this trial
8. Clinically significant abnormalities of full blood count, renal function, or hepatic function:
   a. Elevated liver function tests, including total bilirubin (TBILI)>1.5× the upper limit of normal (ULN; unless subject has documented Gilbert's disease), aspartate aminotransferase (AST) or alanine aminotransferase (ALT)>3.0×ULN.
   b. Subjects with both a creatinine >1.5×ULN and an estimated creatinine clearance of <60 mL/min (using the (using the Cockcroft-Gault equation) c. Hemoglobin <10 g/dL; platelet <100×10⁹/L; absolute neutrophil count <1.5×10⁹/L
9. Any contraindication to MRI and/or Gd, such as allergy or any object that is reactive to strong static magnetic, pulsed-gradient fields including any metallic fragments or foreign body (eg, aneurysm clip(s), pacemakers, electronic implants, shunts)
10. Prior cancers, except successfully treated non-melanoma skin cancer or carcinoma in situ of the cervix, with a ≥5% chance of recurrence within 12 months
11. Immunomodulatory therapy (apart from short courses of corticosteroids) as follows:
   a. Any previous treatment with a B-cell depleting agent
   b. Any previous treatment with alemtuzumab
   c. Treatment with glatiramer acetate or IFNβ within 4 weeks of providing informed consent
   d. Treatment with dimethyl fumarate within 4 weeks of providing informed consent
   e. Treatment with fingolimod within 2 months of providing informed consent
   f. Treatment with natalizumab, methotrexate, azathioprine, or cyclosporine within 6 months of providing informed consent
   g. Treatment with teriflunomide within 12 months of providing informed consent unless patient has completed an accelerated clearance with cholestyramine
   h. Treatment with mitoxantrone, cyclophosphamide, cladribine, rituximab or any other immunosuppressant or cytotoxic therapy (other than steroids) within 12 months of providing informed consent, or determined by the investigator to have residual immune suppression from these treatments
12. Antithymocyte globulin or similar anti-T cell antibody therapy ≤4 weeks before providing informed consent.
13. Female of childbearing potential unwilling to use a highly effective method of contraception (i.e., one that results in pregnancy less than 1% per year when used consistently and correctly), e.g., implants, injectables, combined oral contraceptives, some intrauterine contraceptive devices, sexual abstinence, or a vasectomized partner while undergoing treatment with ATA188 and for 3 months after the last dose.
OR
Men with a female partner of childbearing potential unwilling to use a highly effective contraceptive measure and/or unwilling to refrain from donating sperm while undergoing treatment with ATA188 and for 3 months after the last dose
14. Women who are breastfeeding.
15. Pregnancy.
16. Inability to comply with study procedures.
17. Previous treatment with EBV T-cell therapy.

### Statistical Methods Utilized in the Study

### Analysis Population

All subjects enrolled in the study and who receive any study product will be included in the efficacy and safety populations. The efficacy population will be for the primary efficacy analyses, and all analyses of disposition, demographic and baseline disease characteristics.

In order for a subject to be considered evaluable for the analysis of a DLT, the subject should have either had a DLT during the 35-day DLT assessment window or had completed the 35-day DLT assessment.

### Efficacy Analyses

The descriptive statistics will be provided for the efficacy endpoints, and in addition the continuous efficacy endpoints will be analyzed using regression methods.

### Safety Analyses

Safety assessments will include all related and unrelated AEs. All AEs will be mapped using the Medical Dictionary for Regulatory Activities and graded according to the CTCAE version 4.03. AEs will be summarized by the number and percentage of subjects for whom AEs were reported, serious versus non-serious, and investigator-reported relationship (unrelated, possibly related, related). Descriptive statistics will be used to summarize AE types and frequencies.

### Example 5: CTLs from Healthy Donors Show Improved Effector Function

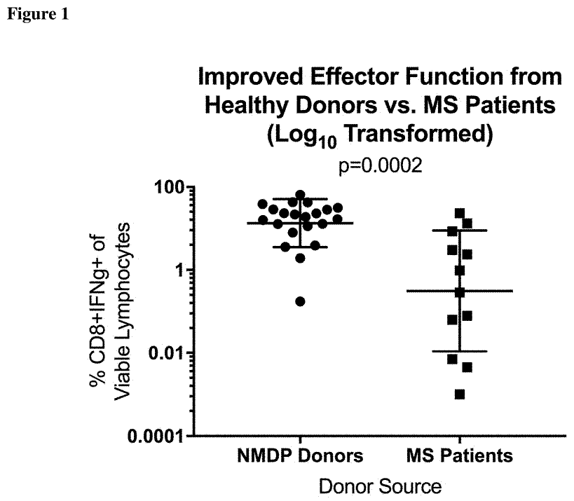

Peripheral blood mononuclear cells (PBMCs) were obtained from of healthy EBV seropositive donors (NMDP Donors) or MS patients. A portion of each of these donor cell samples were used for as the source of expanded CTLs and a portion were used as a source the antigen presenting cells (APCs) which used to stimulate the CTLs. The APCs were transduced with a recombinant, replication-deficient adenovirus encoding a transgene that expresses a polypeptide protein and truncated EBNA1 protein (AdE1-LMPpoly). The polyepitope protein included multiple HLA class I-restricted CD8⁺ T cell epitopes from LMP1 and LMP2 as a “string of beads”. The truncated EBNA1 protein excluded the glycine-alanine repeat sequence which interferes with translation and endogenous processing of this protein and maintains the CD8+ and CD4+ T cell epitopes. The CTL portion of the donor cell sample was co-cultured with the prepared APCs to expand and stimulate CTLs in the sample specific for the EBV epitopes. Following stimulation and generation of CTL comprising products, the CTL batches were tested for effector function by FACs. As seen in FIG. 1, the CTL products generated from healthy donors had a significantly higher percentage of viable lymphocytes that are interferon γ (IFNg) expressing and CD8⁺ compared to CTL products generated from MS patients (Mann Whitney p value of 0.0002). These data show that a more robust CTL product with a higher fraction of effector CD8 T cells and functional IFNg⁺ CTLs is generated when healthy donors are used as the source of CTLs for allogenic transfer compared to when MS patients are used as the source of CTLs for autologous transfer.

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

Those skilled in the art will recognize, or be able to ascertain using no more than routine experimentation, many equivalents to the specific embodiments of the invention described herein. Such equivalents are intended to be encompassed by the following claims.

1. A method of treating or preventing a systemic autoimmune disease in a subject, comprising administering allogeneic cytotoxic T cells (CTLs), wherein said CTLs express one or more EBV-specific T cell receptors, and wherein said CTLs have been induced to proliferate with EBV peptides selected from an EBV EBNA-1 peptide, an EBV LMP1 peptide, an EBV LMP2A peptide, or any combination thereof.

2. The method of claim 1, wherein the EBV peptides are presented on a class I MHC encoded by an HLA allele that is present in the subject, and the EBV-specific T cell receptors specifically bind the EBV peptide/MHC complex.

3. The method of claim 1, wherein the systemic autoimmune disease is selected from rheumatoid arthritis (RA), systemic lupus erythematosus, and Sjögren's syndrome.

4. The method of claim 1, wherein the allogeneic CTLs are obtained from a cell bank.

5. The method of claim 1 comprising:
a) selecting CTLs from a cell bank of allogeneic CTLs, wherein the selected CTLs express one or more EBV-specific T cell receptors, wherein said CTLs have been induced to proliferate with EBV peptides selected from an EBV EBNA-1 peptide, an EBV LMP1 peptide, an EBV LMP2A peptide, or any combination thereof;
b) administering the allogeneic CTLs to the subject.

6. The method of claim 1, wherein the systemic autoimmune disease is selected from RA, systemic lupus erythematosus, and Sjögren's syndrome.

7. A method of treating or preventing a systemic autoimmune disease in a subject, comprising:
a) incubating a sample comprising allogeneic CTLs with antigen-presenting cells (APCs) presenting an EBV peptide, thereby inducing proliferation of peptide-specific CTLs expressing T cell receptors that specifically bind to the EBV peptide presented on a class I MHC;
b) administering the peptide-specific allogeneic CTLs to the subject.

8. The method of claim 7, wherein the class I MHC is encoded by an HLA allele that is present in the subject.

9. A method of treating or preventing a systemic autoimmune disease in a subject, comprising:
a) incubating APCs with a nucleic acid construct encoding for an EBV peptide, thereby inducing the APCs to present an EBV peptide;
b) inducing peptide-specific CTL proliferation by incubating a sample comprising allogeneic CTLs with the APCs, thereby inducing proliferation of CTLs expressing a T cell receptor that specifically binds to the EBV peptide presented on a class I MHC; and
c) administering the peptide-specific allogeneic CTLs to the subject.

10. The method of claim 9, wherein the class I MHC is encoded by an HLA allele that is present in the subject.

11. The method of claim 9, wherein the nucleic acid construct is a viral vector.

12. The method of claim 11, wherein the viral vector is AdE1-LMPpoly.

13. The method of claim 8, wherein the allogeneic CTLs are stored in a cell bank before being administered to the subject.

14. The method of claim 8, wherein the systemic autoimmune disease is selected from RA, systemic lupus erythematosus, and Sjögren's syndrome.

15. The method of claim 8, wherein the sample is incubated with one or more cytokines in step (a).

16. The method of claim 8, wherein the APCs comprise B cells, antigen-presenting T-cells, dendritic cells, or artificial antigen-presenting cells.

17-19. (canceled)

20. The method of claim 16, wherein the artificial antigen-presenting cells are aK562 cells.

21. The method of claim 8, wherein the sample comprises peripheral blood mononuclear cells (PBMCs).

22. The method of claim 1, wherein the EBV peptide comprises a LMP1 peptide, a LMP2A peptide, an EBNA1 peptide, or a fragment thereof.

23-24. (canceled)
